(19) United States
(12) Patent Application Publication
(10) Pub. No.: US 2018/0200368 A1
(43) Pub. Date: Jul. 19, 2018

(54) DISRUPTION OR ALTERATION OF MICROBIOLOGICAL FILMS

(71) Applicant: UNIVERSITEIT GENT, Gent (BE)

(72) Inventors: Kevin BRAECKMANS, Daknam (BE); Tom COENYE, Mariakerke (BE); Joseph DEMEESTER, Gent (BE); Stefaan DE SMEDT, Mariakerke (BE)

(21) Appl. No.: 15/743,729

(22) PCT Filed: Jun. 29, 2016
(86) PCT No.: PCT/EP2016/065073

§ 371 Date: Jan. 11, 2018

(30) Foreign Application Priority Data
Jul. 14, 2015 (EP) 15176559.1
Jul. 14, 2015 (EP) 15176560.9

(51) Int. Cl. A61K 41/00; A61K 9/127; A61P 1/02; A61N 5/06

(52) U.S. Cl. CPC A61K 41/0028; A61K 9/127; A61P 1/02; A61N 2005/067; A61N 2005/0661; A61N 2005/0659; A61N 5/062

(57) ABSTRACT

A method for altering and/or disrupting a microbiological film is described. The method comprises introducing a composition into a microbiological film, the composition comprising nano- or microparticles having an electron density that can couple with a photon wave of electromagnetic radiation. The method also comprises irradiating said microbiological film by said electromagnetic radiation such as to form a vapour bubbles using said nano- or microparticles in said microbiological biofilm, thereby generating a mechanical force for locally altering and/or disrupting said microbiological film when said vapour bubbles expand and/or collapse.

### FIELD OF THE INVENTION

The invention relates to the field of healthcare and biofouling. More specifically, the present invention relates to methods and systems for disrupting of microbiological films, as may for example occur at infected wounds or at certain tissues.

### BACKGROUND OF THE INVENTION

Biofilms are consortia of micro-organisms that form on various (a)biotic surfaces and are implicated in persistent infections, such as in chronic wounds and dental root canals. One of the typical properties of biofilm cells is their decreased sensitivity to antimicrobial agents (AMA) as compared to non-adherent cells. One of the important reasons for antimicrobial resistance of biofilms is hindered penetration of AMA through biofilms. If the rate of antibiotic penetration through a biofilm is decreased, the organisms may initially be exposed to a low concentration of the antibiotic and may have time to mount a defensive response. Hindered diffusion is caused by the fact that cells are often packed together in dense clusters of tens to hundreds of micrometers in size, due to which AMA cannot easily reach deep cell layers. Furthermore, cells are surrounded by a biofilm matrix composed of exopolysaccharides (EPS), proteins and extracellular DNA (eDNA). The biofilm matrix can hinder AMA diffusion due to physicochemical interactions of AMA with matrix constituents. A potential way to improve AMA diffusion to cells in a biofilm is by interfering with the biofilm structure so that cells are released from their protective environment.

To interfere with the biofilm structure, the most common approach is to treat the biofilms with pharmacological compounds. For instance, this can be achieved by interfering with quorum sensing increased or by degrading the EPS matrix (e.g. using the glycoside hydrolase dispersin B) or eDNA (e.g. using deoxyribonuclease I). However, matrix polymers are often stabilized (e.g. stabilization of eDNA with the protein IHF in Burkholderia cenocepacia biofilms) and cannot be easily degraded. Also, as the matrix composition may display quantitative and qualitative variations between different strains, matrix-degrading compounds cannot be broadly applied due to their high specificity. This is corroborated by the fact that in medical settings numerous microbial species may grow within the same biofilm, further increasing the biochemical heterogeneity of the matrix.

Methods based on physical approaches to destabilize the structural integrity of biofilms may be more generally applicable. The use of laser light represents one interesting option. In recent years, the interest in using lasers for killing bacteria has steadily grown. Especially in combination with gold nanoparticles (AuNPs) it has been shown that bacteria can be killed by photothermolysis. Due to plasmon resonances, AuNPs can very efficiently absorb laser light due to which they become heated and cause thermal damage to nearby bacterial cells.

There is still need for good systems and methods for disrupting or altering microbiological films.

### SUMMARY OF THE INVENTION

It is an object of embodiments of the present invention to provide good systems and methods for disrupting microbiological films.

It is an advantage of embodiments of the present invention that the dense structure of microbiological films can be altered, allowing better penetration of antimicrobial agents deep into the microbiological films, such as those present in chronic wound infections.

It is an advantage of embodiments according to the present invention that there is no or limited diffusion of heath so that aspecific effects on healthy tissue caused by heating can be avoided.

It is an advantage of embodiments of the present invention that medicaments can be shielded from interacting with biofilm components, e.g. by incorporation into liposomal formula, and therefore it can be achieved that the medicaments are released at a high dose near the bacteria. The latter can e.g. be obtained by combined action of cell cluster disruption and light triggered release of medicaments from liposomes by laser-induced VNBs, close to the bacteria.

It is an advantage of embodiments of the present invention that the methods are generally applicable, independent on the composition of the biofilm or the presence of multiple microbial species within the same biofilm.

It is an advantage of embodiments according to the present invention that the mechanical disturbance of the structure occurs uniformly through the three dimensional biofilm such that the antimicrobiotic products can enter deeply into the biofilm.

It is an advantage that the vapour bubble size can be tuned using the laser intensity and thereby that the degree in which the biofilm is disturbed or disrupted can be tuned. The latter is advantageous as for some applications, one only wants to loosen the film rather than to physically disrupt it completely, whereas in other applications one wants to disrupt the film completely.

The above objective is accomplished by a method and device according to the present invention.

In one aspect, the present invention relates to a method for altering or disrupting a microbiological film, the method comprising introducing a composition into a microbiological film, the composition comprising nano- or microparticles having an electron density that can couple with a photon wave of electromagnetic radiation; and irradiating said microbiological film by said electromagnetic radiation such as to form vapour bubbles using said plasmonic nanoparticle in said microbiological film, e.g. bacterial biofilm, thereby generating a mechanical force for locally altering or disrupting said microbiological film when said vapour nanobubble expands and/or collapses. The structure of the biofilm is altered, e.g. loosened, and/or disrupted from the inside thereof, thus allowing a more efficient disruption and for example also allowing the antimicrobial agent to diffuse better into the composition. It is an advantage that the nano- or mircoparticles do not need to be deposited as a film, but can diffuse through the water channels in the biofilm, thus allowing action from the inside of the biofilm. While the mechanical force or the antimicrobial agent might each kill bacteria in the biofilm in isolation, a strong, synergistic effect is achieved by the combination. The bacterial biofilm may comprise a hydrated biofilm, e.g. may have a water content taking up at least 50% of the total volume, e.g. at least 70% of the total volume, such as to allow said nanobubbles to form. The mechanical force may be induced by expanding and/or collapsing of the vapour bubbles. The microbiological film may be a bacterial biofilm.

Irradiating said microbiological film by said pulse of electromagnetic radiation may comprise irradiating the microbiological film, e.g. bacterial biofilm, by a pulsed irradiation source, said pulse of electromagnetic radiation having a pulse length in the range of 10 ns down to 10 fs.

The microbiological film may be any group of microorganisms in which cells stick to each other and cells adhere to a surface.

Locally altering or disrupting the microbiological film may comprise disrupting the film such that cells become loosened from each other and/or from the surface.

Irradiating said microbiological film by said pulse of electromagnetic radiation may comprise irradiating the bacterial biofilm by said pulse of electromagnetic radiation having a fluence of at least 1 mJ/cm². It is an advantage that the light energy is delivered in such a short time span so that the nanoparticle heats quickly before heat diffusion can occur.

Said nano- or microparticles may be diffusible particles. Diffusible particles are particles that can diffuse into the microbiological film through Brownian motion.

Said nano- or microparticles may be plasmonic nano-or microparticles.

Said nano or microparticles may comprise any or a combination of gold, silver or titanium nano or microparticles.

Said nano- or microparticle may comprise carbon-based nano or microparticles.

The carbon-based particles may be graphene oxide nano or microparticles, carbon nanotubes, carbon dots or fullerenes.

It is an advantage of embodiments that the materials used for the nano or microparticles are readily available, have been well studied and have good biocompatibility.

Said plasmonic nanoparticle may be surface functionalized for improving colloidal stability and/or for avoiding aggregation and/or for targeting (e.g. targeting antibodies). It is an advantage of embodiments of the present invention that a good dispersion and/or penetration of the nano- or microparticles into the biofilm can be obtained.

Irradiating said bacterial biofilm by said pulse of electromagnetic radiation may comprise using a pulsed laser to generate said pulse.

It is an advantage of embodiments of the present invention that a well delineated and efficient energy transfer can be performed, towards the nano- or mircoparticles.

Said irradiation may be irradiation with a wavelength in a range from ultraviolet to infrared.

Radiation in the near infrared region, e.g. between 700 and 1300 nm, may be used.

Said antimicrobial agents may be encapsulated in a nano- or micro carrier, which may also be referred to as a container.

The nano- or micro carrier may comprise liposomes or lipids or polymer-based nano- or mircoparticles (e.g. PLGA).

The nano- or microparticles may be linked to the liposomes or lipids or polymer-based nanoparticles. In some embodiments the nano- or microparticles may be encapsulated in the containers formed by the liposomes or lipids or polymer-based nanoparticles. In other embodiments the nano- or microparticles are conjugated to the containers.

The containers may have an outer diameter less than or equal to 500 nm.

Introducing the composition into the microbiological film may comprise diffusing said composition into a bacterial biofilm infecting a wound.

Introducing the composition into the microbiological film may comprise diffusing said composition into a bacterial biofilm infecting a dental root canal.

In some embodiments the nano- or microparticles may be oil or fluorocarbon drops and the vapour bubbles may be vapour bubbles formed from the oil or fluorocarbon drops themselves. The oil drop may comprise absorbing material so as to be able to absorb electromagnetic wave energy. Such absorbing material may be an inherent characteristic of the oil or fluorocarbon drop or may be a characteristic of a material added to the oil or fluorocarbon drop.

Said irradiation may comprise irradiating said microbiological film for subsequently forming first vapour bubbles and at least second vapour bubbles stemming from the same batch of nano- or microparticles or even from the same nano- or microparticles.

The present invention also relates to a composition for introducing into a bacterial biofilm, said composition comprising antimicrobial agents and nano- or microparticles having an electron density that can couple with a photon wave in a pulse of electromagnetic radiation such as to generate a vapour bubble upon irradiation by said pulse of electromagnetic radiation.

The nano-or microparticles may be silver particles, titanium particles or carbon-based particles. The carbon-based particles may be graphene oxide nano or microparticles, carbon nanotubes, carbon dots or fullerenes.

The antimicrobial agents may be encapsulated in containers. The containers may comprise liposomes or lipids or polymer-based nanoparticles. The nano or microparticles may be linked to the liposomes or lipids or polymer-based nanoparticles. In some embodiments the nano- or microparticles may be encapsulated in the containers formed by the liposomes or lipids or polymer-based nanoparticles. In other embodiments the nano- or microparticles are conjugated to the containers. The container may have an outer diameter less than or equal to 500 nm.

The plasmonic nanoparticle may be a gold, silver, titanium, graphene oxide, fullerene or other carbon based tube nanoparticle having a diameter in the range of 10 nm to 100 nm.

According to embodiments of the present invention, the antimicrobial agents may be bound directly on the nano- and microparticles.

The present invention furthermore relates to the use of a composition as described above for diffusion into a microbiological film.

The present invention also relates to a composition for treatment of wounds, said composition comprising disinfectants and nano or microparticles having an electron density that can couple with a photon wave in a pulse of electromagnetic radiation. The desinfectants may in some embodiment be any of povidone iodine [PVP-I], peroxide-based preparations or chlorhexidine. The disinfectants may be encapsulated in containers. The containers may comprise liposomes or lipids or polymer-based nanoparticles. The nano or microparticles may be linked to the liposomes or lipids or polymer-based nanoparticles. In some embodiments the nano- or microparticles may be encapsulated in the containers formed by the liposomes or lipids or polymer-based nano- or mircoparticles. In other embodiments the nano- or microparticles are conjugated to the containers.

The nano-or microparticles may be silver particles, titanium particles or carbon-based particles. The carbon-based particles may be graphene oxide nano or microparticles, carbon nanotubes, carbon dots or fullerenes.

The present invention furthermore relates to a composition for treatment of apical periodontitis, said composition comprising antimicrobial agents and nano- or microparticles having an electron density that can couple with a photon wave in a pulse of electromagnetic radiation. The antimicrobial agents may be NaOCl.

The nano-or microparticles may be silver particles, titanium particles or carbon-based particles. The carbon-based particles may be graphene oxide nano or microparticles, carbon nanotubes, carbon dots or fullerenes.

The antimicrobial agents may be encapsulated in containers. The containers may comprise liposomes or lipids or polymer-based nanoparticles. The nano or microparticles may be linked to the liposomes or lipids or polymer-based nanoparticles. In some embodiments the nano- or microparticles may be encapsulated in the containers formed by the liposomes or lipids or polymer-based nanoparticles. In other embodiments the nano- or microparticles are conjugated to the containers. The nano-or microparticles may be silver particles, titanium particles or carbon-based particles. The carbon-based particles may be graphene oxide nano or microparticles, carbon nanotubes, carbon dots or fullerenes.

Particular and preferred aspects of the invention are set out in the accompanying independent and dependent claims. Features from the dependent claims may be combined with features of the independent claims and with features of other dependent claims as appropriate and not merely as explicitly set out in the claims.

These and other aspects of the invention will be apparent from and elucidated with reference to the embodiment(s) described hereinafter.

### BRIEF DESCRIPTION OF THE DRAWINGS

FIG. 1 illustrates the effect of using nano- or mircoparticles for disrupting biofilms through laser-induced explosive nanobubbles, according to an embodiment of the present invention.

FIG. 2 illustrates (A) an optical setup for performing a method according to an embodiment of the present invention, (B) a comparison of the heat dissipation when using a laser fluence below and above the VNB threshold, (C) pictures illustrating disruption induced using vapour nanobubbles, illustrating features of embodiments of the present invention.

FIG. 3 illustrates in pictures A to C confocal images of Au nanoparticles that penetrate biofilms of (A) Burkholderia multivorans, (B) Pseudomonas aeruginosa and (C) Staphylococcus aureus biofilms, as can be obtained using a method according to the present invention.

FIG. 4 illustrates the biofilm before laser illumination, during vapour nanobubble formation and after vapour nanobubble formation, illustrating features of methods according to the present invention for biofilms of (A) Burkholderia multivorans, (B) Pseudomonas aeruginosa and (C) Staphylococcus aureus.

FIG. 5 illustrates a comparison of the effect of different treatments, illustrating advantages of combined vapour nanobubbles generation and delivery of an antimicrobial agent for biofilms of (A) Burkholderia multivorans, (B) Pseudomonas aeruginosa and (C) Staphylococcus aureus according to embodiments according to the present invention.

FIG. 6 illustrates the effect of subsequent laser pulses on the disruption of a microbiological film, according to an embodiment of the present invention.

FIG. 7 illustrates the effect of subsequent laser pulses and delivery of an antimicrobial agent, illustrating advantages of combined subsequent vapour nanobubble formation and delivery of an antimicrobial agent according to embodiments of the present invention.

The drawings are only schematic and are non-limiting. In the drawings, the size of some of the elements may be exaggerated and not drawn on scale for illustrative purposes.

Any reference signs in the claims shall not be construed as limiting the scope.

In the different drawings, the same reference signs refer to the same or analogous elements.

### DETAILED DESCRIPTION OF ILLUSTRATIVE EMBODIMENTS

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. The dimensions and the relative dimensions do not correspond to actual reductions to practice of the invention.

Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

Moreover, the terms top, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Where in embodiments of the present invention reference is made to a microbiological film, reference—according to its IUPAC definition—may be made to an aggregate of microorganisms in which cells that are frequently embedded within a self-produced matrix of extracellular polymeric substance adhere to each other and/or to a surface. It is to be noticed that a biofilm is a system that can be adapted internally to environmental conditions by its inhabitants. The self-produced matrix of extracellular polymeric substance, which is also referred to as slime, is a polymeric conglomeration generally composed of extracellular biopolymers in various structural forms.

Alternatively where in embodiments of the present invention reference is made to a microbiological film, reference may be made to any group of microorganisms in which cells stick to each other and often these cells adhere to a surface. These adherent cells may be frequently embedded within a self-produced matrix of extracellular polymeric substance (EPS).

Where in embodiments of the present invention reference is made to altering and/or disrupting of a microbiological films, reference is made to both situations where one alters the density of the biological film, e.g. decreases the density of the microbiological film or increases the space between the cells without releasing cells from the biofilm or where one at least partially or completely disrupts the film.

Where reference is made to vapour bubbles, reference is made to vapour bubbles having a diameter in the range 10 nm to 100 μm. Such vapour bubbles may be water vapour bubbles in some applications, although embodiments are not limited thereto and the vapour bubbles also can be created in or from e.g. oil drops.

Where reference is made to diffusible particles, reference is made to particles that are able to diffuse into the microbiological film through Brownian motion. At least 10%, e.g. at least 30%, e.g. at least 50% of the particles may be able to diffuse into the microbiological film, e.g. diffuse into the microbiological film over at least 1 μm.

In a first aspect, the present invention relates to a method for altering and/or disrupting a microbiological film. The microbiological film may for example be a bacterial biofilm, but embodiments are not limited thereto. The present method advantageously finds its application in for example wound healing and apical periodontitis, although embodiments are not limited thereto.

Whereas in embodiments of the present invention reference is made to disruption or altering of microbiological films, such as bacterial films, in healthcare applications, the present invention is equally applicable to biofouling applications, such as for example disruption or altering of films in industrial applications (e.g. brewing, water cleaning, . . . )

According to embodiments of the present invention, the method comprises introducing a composition into a microbiological film, whereby the composition comprises nano- or microparticles having an electron density that can couple with a photon wave of electromagnetic radiation. The nano- or microparticles may be diffusible nano- or microparticles. The nano- or microparticles may be metal particles, such as for example gold nano- or mircoparticles, also referred to as AuNP, silver nano- or mircoparticles, titanium nano- or mircoparticles, or may be carbon-based nano- or mircoparticles, such as for example graphene oxide based particles or carbon nano- or mircoparticles like carbon nanotubes or carbon dots, or may for example be fullerenes. In alternative embodiments, the nano- or microparticles may be oil drops comprising an absorbing material.

The nano- or microparticles fulfil the requirement of having an electron density that can couple with a photon wave of electromagnetic radiation. Different surface charges may be applied, such as for example the structure may be anionic, neutral or cationic. The particles may be functionalized with ligands, such as poly-ethylene glycol that confer colloidal stability in biological tissues. The nano- or microparticles may have a diameter between 1 nm and 1 μm, e.g. 20 to 200 nm.

In some embodiments, the particles may have a functionalized surface. Such surface functionalization may be any suitable surface functionalization such as for example for improving colloidal stability, for obtaining a certain surface charge, for coupling of antimicrobial agents, for targeting, . . . In some particular examples, such polyethylene amine (PEI), polyethyleneglycol, polysaccharides, lectins, antibodies, peptides, . . .

Obtaining a composition into a microbiological film may include allowing the composition to diffuse into the microbiological film following topical administration (dispensing, flowing over) or actively depositing the composition into the microbiological film, such as by injecting or by ballistically propelling the composition into the microbiological film.

The method also comprises, according to embodiments of the present invention, irradiating the microbiological film by said electromagnetic radiation such as to form a vapour bubble using the nano- or microparticle in the bacterial biofilm. In some embodiments, the vapour bubbles may be water vapour bubbles caused by heating of water around the nano- or microparticles. In other embodiments the vapour bubbles may be created from the nano- or microparticles themselves, for example when oil drops are used, the vapour bubbles may be oil-based vapour bubbles. The irradiation may advantageously be a pulsed irradiation, although embodiments of the present invention are not limited thereto and in principle also a continuous wave irradiation could be used. In the advantageous embodiments wherein pulsed irradiation is used, the pulse may have a duration in the range 10 ns down to 0.1 ns or down to 0.1 ps. The fluence may be adapted depending on the pulse duration. In one example, the fluence may be at least 10 or tens mJ per pulse. The wavelength of the radiation used may range from UV to the IR region. In some applications, the wavelength range of the radiation used may be in the near infrared. One or more pulses could be used for inducing the effect.

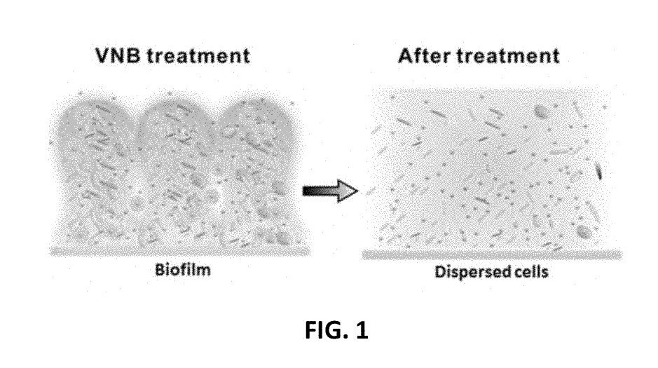

The irradiation thereby is performed such that the heating of the nano- or mircoparticles results in the generation of a mechanical force for locally altering or disrupting said microbiological film when said vapour nanobubble expands and/or collapses. It is to be noticed that the vapour bubbles do not need to explode or implode but that also the fact of expanding or increasing their volume may cause an altering or disrupting effect. The process is schematically illustrated with reference to FIG. 1 whereby the effect of generating vapour bubbles, in the example shown water vapour nanobubbles (VNBs), by pulsed laser irradiation is illustrated. In the example shown in FIG. 1, a short laser pulse (<10 ns) causes rapid heating of AuNP to very high temperatures due to which a vapour bubble emerges around the AuNP in the tissue. The size of the vapour bubbles can vary from ten to several hundred nm depending on the laser fluence. When the thermal energy of the AuNP is consumed, the vapour bubble violently collapses and causes local damage by high-pressure shock waves. Due to the extremely short lifetime of VNBs (typically<10 μs), the diffusion of heat from the AuNP into the environment is negligible so that almost all energy of the irradiated AuNP is converted to mechanical energy (expansion of the VNB) without heating of the environment. This property makes VNBs an interesting phenomenon to cause local mechanical damage, without causing thermal damage to healthy tissue, which is a concern in classic hyperthermia therapies. It provides a good alternative for direct heating of biofilms, where there is a risk of causing aspecific thermal damage to the surrounding healthy tissue.

In one set of embodiments, the method according to embodiments of the present invention furthermore may comprise nanomedicine formulations of antimicrobial agents in combination with vapour bubbles. Such a release can be done by applying both the step of inducing disruption of a microbiological film using vapour bubbles and providing nanomedicines to the infected region. The latter results in the advantageous effect that the nanomedicines can get more easily to the infected region, since the microbiological film is disrupted and cannot act as a diffusion barrier. It thus is found that by physically altering and/or disrupting the microbiological clusters, nanomedicines can more easily reach deep cell layers, resulting in an improved treatment efficacy.

An example of such nanomedicines are liposomes encapsulating AMA. One example is Arikayce, a liposomal formulation of amikacin for inhalation by CF patients that is currently in clinical phase III trials. Another example is Repithel, a liposomal formulation of PVP-I that is used for the treatment of wound infections.

In yet another embodiment, the irradiation used for creating vapour bubbles using nano- or mircoparticles is also used for releasing the AMA from the nanomedicines close to the bacteria or infected region. In such embodiments the nanomedicines may be conjugated to the nano- or mircoparticles that create the vapour bubbles. This will improve penetration of AMA to the bacteria, resulting in a more effective treatment of biofilm infections.

In some embodiments, the method further encloses generating a plurality of subsequent vapour bubbles generated using the same nano- or microparticle that is introduced in the object. The latter may have as an advantage that improved disruption is obtained of the microbiological film, allowing better penetration of antimicrobial agents. Multiple subsequent bubbles thus can be formed from the same particle by repeated or prolonged irradiation, e.g. laser irradiation. In some embodiments, a plurality of short pulses having a pulse duration of less than 10 ns may be used. It is an advantage that diffusion of heat is limited so that there is no or only limited damage to surrounding, untargeted parts of the object. In one aspect, the present invention also relates to a composition for introducing into a microbiological film, said composition comprising an antimicrobial agents and nano- or microparticles having an electron density that can couple with a photon wave in a pulse of electromagnetic radiation such as to generate vapour bubbles upon irradiation by said pulse of electromagnetic radiation. In some embodiments, the antimicrobial agents may be encapsulated in containers. The containers may comprise liposomes, lipids and polymer based nano- or mircoparticles and the nano- or microparticle may be linked thereto. The nano- or microparticles may be encapsulated in the containers or may be bound to a surface thereof. The container may have an outer diameter less than or equal to 1 μm. Alternatively, the antimicrobial agents also may be bound directly to the nano- or microparticles. The nano- or microparticles may be plasmonic particles. It may be diffusible particles. It may be one or a combination of gold, silver, titanium, graphene oxide or carbon based tube nano- or mircoparticles having a diameter in the range of 20 nm to 200 nm. Further features and advantages may be as described for the composition or parts thereof in the first aspect.

One example of an application is the treatment of apical periodontitis. Microbiological films can form in the dental root canal. Infection may lead to an abscess and potentially to loss of the tooth. In view of the complex structure of the root canal, embodiments of the present invention can advantageously make use of e.g. diffusible particles that allow diffusing towards and into the microbiological film and therefore allow efficient disruption of the film. The present invention therefore also relates to a composition for use in the treatment of apical periodontitis, said composition comprising an antimicrobial agent encapsulated in a container and a plasmonic nanoparticle having an electron density that can couple with a photon wave in a pulse of electromagnetic radiation.

Another example of an application is the treatment of chronic wounds. Healing of wounds can be tremendously slowed down by the presence of microbiological films. Embodiments of the present invention allow an efficient treatment since the nano- or mircoparticles diffuse into the microbiological film and disrupt the film from the inside out. The present invention therefore also relates to a composition for use in the treatment of wounds, said composition comprising an antimicrobial agent encapsulated in a container and a plasmonic nanoparticle having an electron density that can couple with a photon wave in a pulse of electromagnetic radiation.

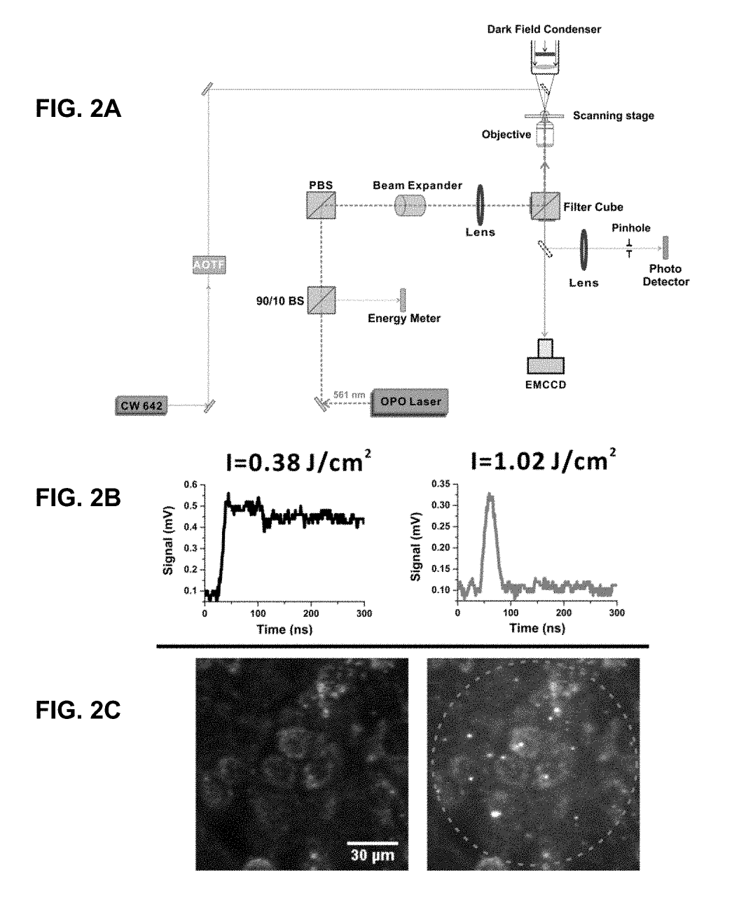

By way of illustration, embodiments of the present invention not being limited thereto, an example of a study of vapour bubbles induced biofilm disruption and AMA treatment is described. By way of illustration, first an optical set-up that can be used for treatment of biofilms by laser-induced vapour bubbles is described. The optical set-up has an optical design as shown in FIG. 2A. The setup is built around an epi-fluorescence microscope equipped with an electronic sample stage and dark field condenser. In a particular example the system is adapted for detecting transmitted light of a continuous wave laser (642 nm) using a fast photodiode. Such an exemplary set-up has the capability to discriminate AuNP heating from VNB formation, respectively. At high intensity of the pulsed laser, VNBs will be induced around the AuNPs which causes refraction of the transmitted CW laser beam during the lifetime of the VNB (typically 10 s to 100 s of nanoseconds). However, as all heat energy is converted to mechanical energy, there is no heating of the surrounding tissue. As a result, after VNB generation the tissue is in its original (thermal) state and the transmitted CW laser beam is no longer refracted. This can be seen in FIG. 2B (curve on the right hand side) where the observed CW laser intensity is the same before and after VNB generation. The vapour bubbles photothermal traces show that there is no heat transferred into the environment as the baseline is identical before and after vapour bubbles generation. On the other hand, when low intensity pulsed laser light is used, VNBs are not created and heat from the AuNP will diffuse into the surrounding tissue. This can be seen from the intensity trace in FIG. 2B (curve on the left hand side), illustrating a typical photothermal signal of AuNP excited by a laser pulse with a fluence below the vapour nanobubble threshold and showing fast heating of the AuNP with a long tail arising from heat diffusion into the surrounding medium. In addition, the exemplary optical set-up has the capability to detect VNBs by dark-field microscopy on a CCD camera, as illustrated in FIG. 2C. HeLa cells are shown, whereby the vapour bubbles are visible as bright spots of scattered light. The circle illustrates the laser illumination area of approximately 100 μm diameter. An electronic sample stage is used to scan the sample over the laser beam to automatically treat large areas of the sample.

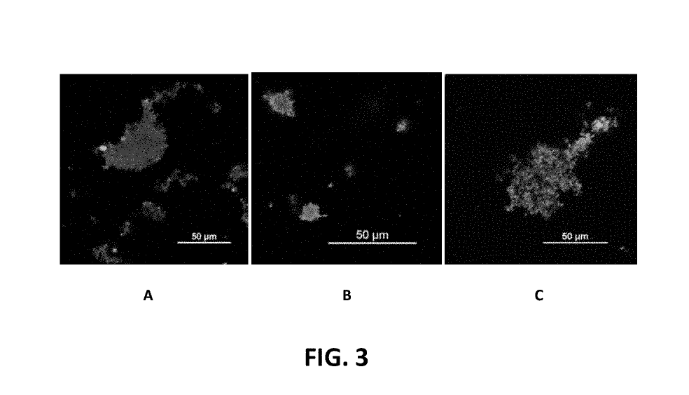

In the study, the synergistic effect of vapour nanobubble mediated cluster disruption and antibiotic treatment was evaluated for 3 types of bacteria. As gram-negatives, Burkholderia multivorans LMG18825 and Pseudomonas aeruginosa LESB58 were selected. Staphylococcus aureus Mu50, often implicated in skin and soft tissue infections, was chosen as a challenging representative of gram-positive bacteria. AuNPs were evaluated for achieving efficient loading and disruption of biofilm clusters. AuNP penetration was assessed using confocal microscopy and an example image is shown in FIG. 3, showing 70 nm AuNPs that have penetrated into bacterial clusters of all types of studied biofilms((A) Burkholderia multivorans, (B) Pseudomonas aeruginosa and (C) Staphylococcus aureus). The Bacteria are stained with SYTO59 and the gold particles were detected by reflection of the laser light.

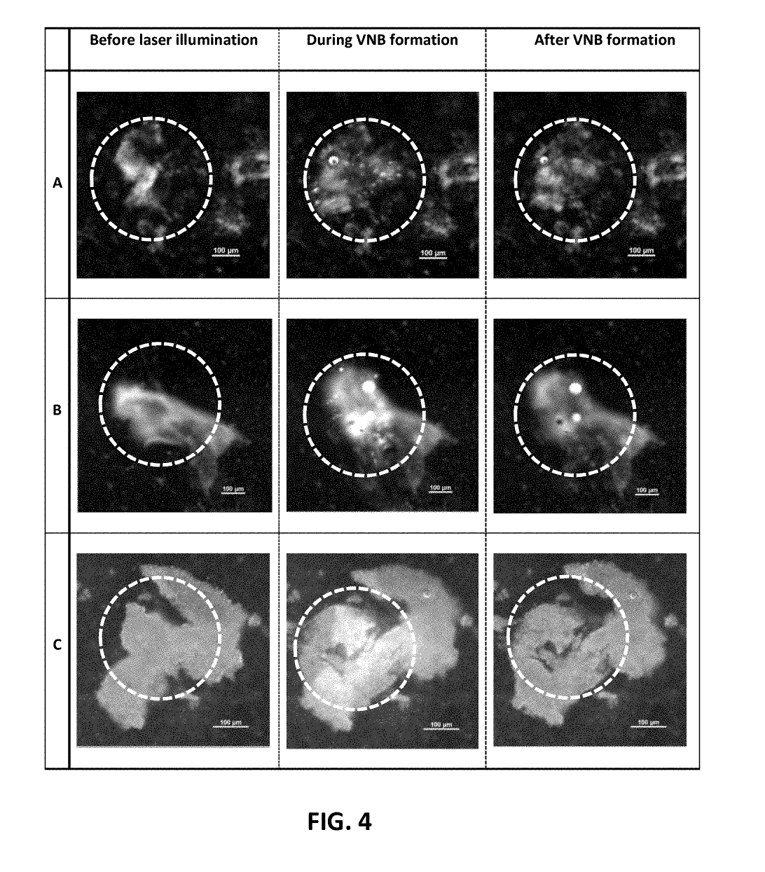

Biofilms loaded with the various types of AuNPs were treated with pulsed laser light to generate VNBs. Cluster disruption is apparent by comparing the volume of bacterial clusters from microscopy images before and after laser treatment. This is illustrated in FIG. 4, showing darkfield microscopy images of cell clusters containing AuNPs (not visible in the picture) before, during, and after laser treatment. The laser treatment in this example is the application of a single nanosecond pulse of a 561 nm laser to each location in the biofilm. The biofilms shown are (A) Burkholderia multivorans, (B) Pseudomonas aeruginosa and (C) Staphylococcus aureus. The white circle marks the illumination area.

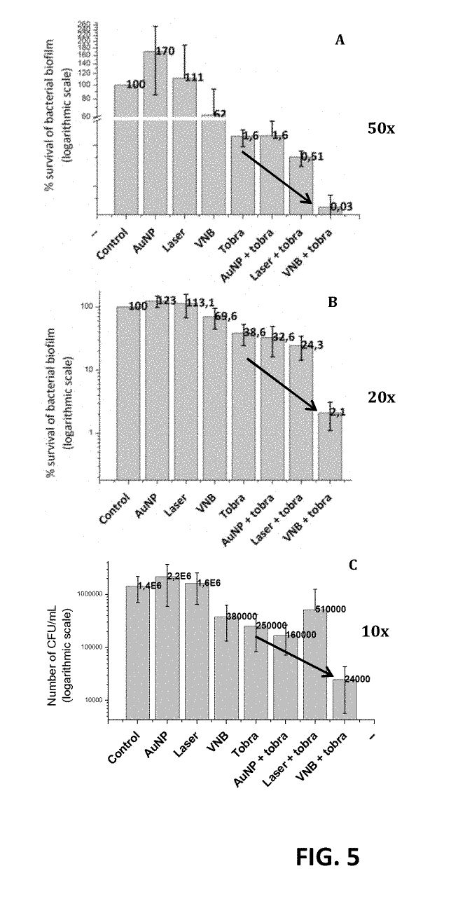

In further experiments, the synergistic effect of VNB biofilm cluster disruption with relevant antibiotics or disinfectants is evaluated. The biofilm first received a laser treatment for cluster disruption, followed by treatment with tobramycin. Proper controls were taken into account: untreated control, only AuNPs, only laser treatment, VNBs only, AuNPs with tobramycin. Each location in the biofilm received a single 7 ns pulse of 561 nm laser light (2.8 J/cm2) followed by Tobramycin treatment at 32 μg/mL, 16 μg/mL and 1024 μg/mL for Burkholderia multivorans, Pseudomonas aeruginosa and Staphylococcus aureus biofilms, respectively. The bacterial viability following vapour nanobubble generation and antibiotic treatment is shown in FIG. 5 The different experiments are as follows:

The control is a 0.9% NaCl solution, AuNP indicates only the addition of 70 nm spherical gold nanoparticles, Laser represents that only a laser treatment is performed in the absence of AuNP or antibiotics, VNB represents the addition of AuNPs with subsequent laser treatment creating vapour nanobubbles, Tobra represents tobramycin treatment for 24 hours at 37° C., AuNP+tobra represents addition of AuNPs with subsequent tobramycin treatment, Laser+tobra represents laser and subsequent tobramycin treatment, VNB+tobra illustrates addition of AUNPs with subsequent laser treatment followed by tobramycin treatment.

As can be seen from the results in FIG. 5, in Burkholderia multivorans biofilms, the use of VNBs showed an increased eradication potency of tobramycin by˜50 times.

The same trend was observed in Pseudomonas aeruginosa and Staphylococcus aureus biofilms, with the combined VNB-tobramycin treatment leading to a CFU decrease˜20 times and˜10 times higher as compared to tobramycin alone, respectively.

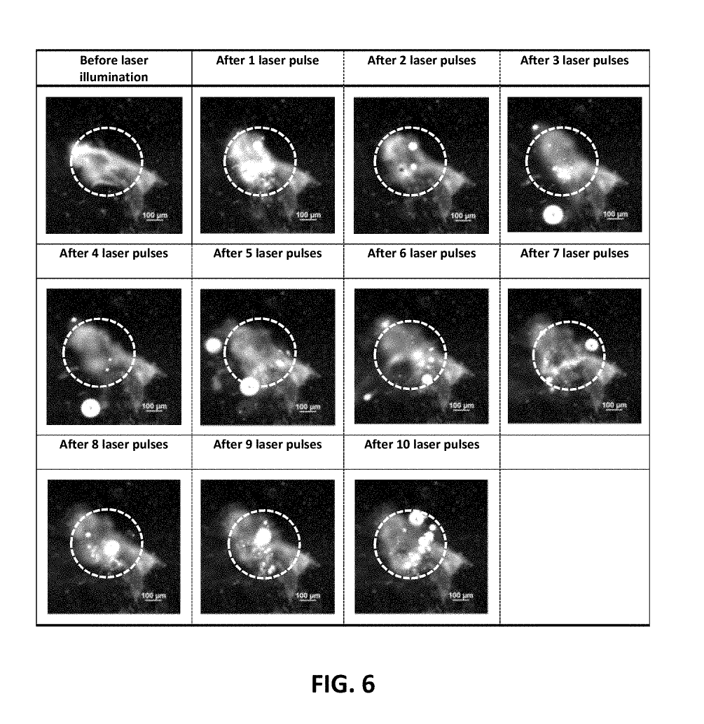

In another experiment, the effect of applying different laser pulses on the biofilm was evaluated. A Pseudomonas aeruginosa biofilm was treated with 10 laser pulses of 7 ns of 561 nm laser light (2.8J/cm²) instead of 1 laser pulse per location. As can be seen in FIG. 6, the biofilm becomes gradually more disrupted with each laser pulse. The images shown are taken by dark field microscopy. The white circle marks the illumination area.

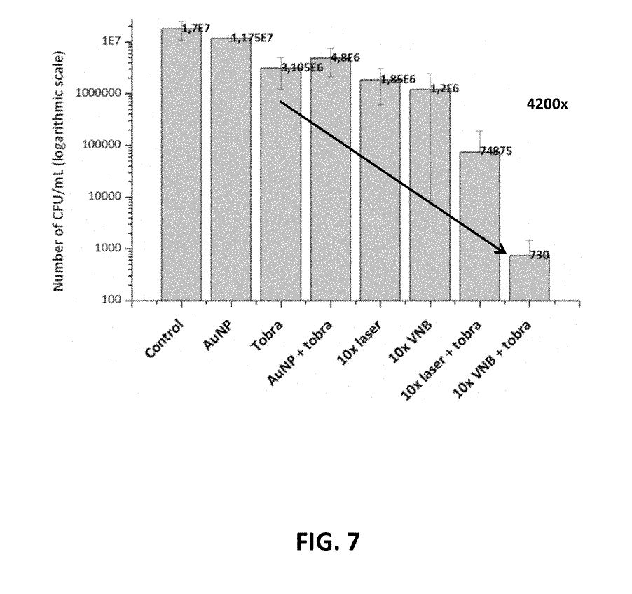

FIG. 7 illustrates the Bacterial viability of a Pseudomonas aeruginosa biofilm following 10 times VNB formation per location in the biofilm and subsequent tobramycin treatment. The control is a 0.9% NaCl solution, AuNP indicates only the addition of 70 nm spherical gold nanoparticles, Tobra represents tobramycin treatment for 24 hours at 37° C., AuNP+tobra represents addition of AuNPs with subsequent tobramycin treatment, 10×laser represents 10 times a pulsed laser treatment, 10×VNB represents the addition of AuNPs with subsequent laser treatment (10×) creating vapour nanobubbles, 10×laser+tobra represents 10×laser illumination and subsequent tobramycin treatment, 10×VNB+tobra illustrates addition of AUNPs with subsequent laser illumination and subsequent tobramycin treatment.

It is found that 10×laser treatment and subsequent addition of tobramycin results in a CFU decrease˜4000 times more in comparison with treatment with tobramycin alone (FIG. 7).

By way of illustration, a further illustration of features and embodiments of the present invention is shown with reference to a further study. In this further study it is evaluated what the effect is of providing the nanomedicines simultaneously with the disruption of the film.

In the present example, selected AMA (biofilm-dependent) are incorporated into liposomes made from 1,2-dipalmitoyl-sn-glycero-3-phosphocholine (DPPC), 1,2-dioleoyl-sn-glycero-3-phosphocholine (DOPC), 1,2-dipalmitoyl-sn-glycero-3-phosphoglycerol sodium salt (DPPG) and cholesterol. AMA loaded liposomes were prepared by a dehydration-rehydration method.

Spontaneous release of AMA from liposomes close to the bacteria is difficult to achieve and highly biofilm-dependent. Therefore, it was studied if the disruptive VNBs can induce efficient triggered release of AMA from liposomes. Liposomes were functionalized with cationic AuNPs (electrostatic adsorption).

Triggered release of liposomes by VNBs were tested in vitro using a calcein release assay. Calcein was incorporated into the liposomes at a high concentration so that its fluorescence was quenched. Upon release, calcein was diluted in the buffer and the fluorescence intensity increased. At the end of the measurement, Triton® X-100 was added to dissolve any remaining liposomes and to complete the calcein release. Thus, for different laser settings and different concentrations of AuNPs the efficiency of release were quantified.

1.-15. (canceled)

16. A method for disrupting and/or altering a microbiological film, the method comprising:
introducing a composition into a microbiological film, the composition comprising nano- or microparticles having an electron density that can couple with a photon wave of electromagnetic radiation; and
irradiating said microbiological film by said electromagnetic radiation such as to form vapour bubbles using said nano- or microparticles in said microbiological film, thereby generating a mechanical force for locally altering or disrupting said microbiological film as the consequence of the expansion and/or collapse of said vapour bubbles.

17. The method according to claim 16, wherein irradiating said microbiological film by said pulse of electromagnetic radiation comprises irradiating the microbiological film by a pulsed irradiation source, said pulse of electromagnetic radiation having a pulse length in the range of 10 ns down to 10 fs.

18. The method according to claim 16, wherein said microbiological film is any group of microorganisms in which cells stick to each other and cells adhere to a surface.

19. The method according to claim 18, wherein locally altering or disrupting said microbiological film comprises disrupting the film such that cells become loosened from each other and/or from the surface.

20. The method according to claim 16, wherein said nano- or microparticles are diffusible and/or plasmonic nano- or microparticles and/or wherein said nano-or microparticles comprise any or a combination of gold particles, silver particles, titanium particles or carbon-based particles.

21. The method according to claim 16, wherein said nano or microparticles are surface functionalized for improving colloidal stability and/or avoiding aggregation and/or for targeting moieties and/or for targeting antibodies.

22. The method according to claim 16, wherein said composition comprises antimicrobial agents and wherein said method comprises enhancing penetration of the antimicrobial agents in the microbiological film.

23. The method according to claim 22, wherein said antimicrobial agents are encapsulated in a nano- or micro carrier.

24. The method according to claim 23, wherein said nano- or micro carrier comprises liposomes and/or wherein said nano or microparticles are linked to said liposomes.

25. The method according to claim 16, wherein said nano- or microparticles are oil drops and wherein said vapour bubbles are formed from said oil drops.

26. The method according to claim 16, wherein said irradiation comprises irradiating said microbiological film for subsequently forming first vapour bubbles and at least second vapour bubbles stemming from the same batch of nano- or microparticles.

27. A composition for introducing into a microbiological film, said composition comprising antimicrobial agents and nano- or microparticles being silver particles, titanium particles or carbon-based particles, said nano or microparticles having an electron density that can couple with a photon wave in a pulse of electromagnetic radiation.

28. A composition according to claim 27, wherein said nano or microparticles have an electron density that can couple with a photon wave in a pulse of electromagnetic radiation such as to generate a vapour bubble upon irradiation by said pulse of electromagnetic radiation.

29. The composition according to claim 27, wherein said antimicrobial agents are encapsulated in containers comprising liposomes, and wherein said nano- or microparticles are linked to said liposomes.

30. A composition for treatment of wounds, said composition comprising disinfectants and nano- or microparticles being silver particles, titanium particles or carbon-based particles, said nano or microparticles having an electron density that can couple with a photon wave in a pulse of electromagnetic radiation.

31. A composition according to claim 27 for treatment of apical periodontitis.
